(19) United States
(12) Patent Application Publication
(10) Pub. No.: US 2002/0002345 A1
(43) Pub. Date: Jan. 3, 2002

(54) DEVICE AND THERAPEUTIC METHOD FOR TREATMENT OF THE HEART OR PANCREAS

(76) Inventor: ERNEST H. MARLINGHAUS, BOTTIGHOFEN (CH)

Correspondence Address: WESLEY W WHITMYER JR, ST ONGE STEWARD JOHNSTON & REENS, 986 BEDFORD STREET, STAMFORD, CT, 06905-5619, US

(21) Appl. No.: 09/242,655

(22) PCT Filed: Aug. 22, 1997
(86) PCT No.: PCT/DE97/01829

(30) Foreign Application Priority Data
Aug. 22, 1996 (DE) 196 33 985.5

(51) Int. Cl. A61B 17/22

(52) U.S. Cl. 601/2

(57) ABSTRACT

What is described here is a therapeutic method and a device for treatment of the hear or the pancreas, for instance, operating on focussed ultrasonic waves.

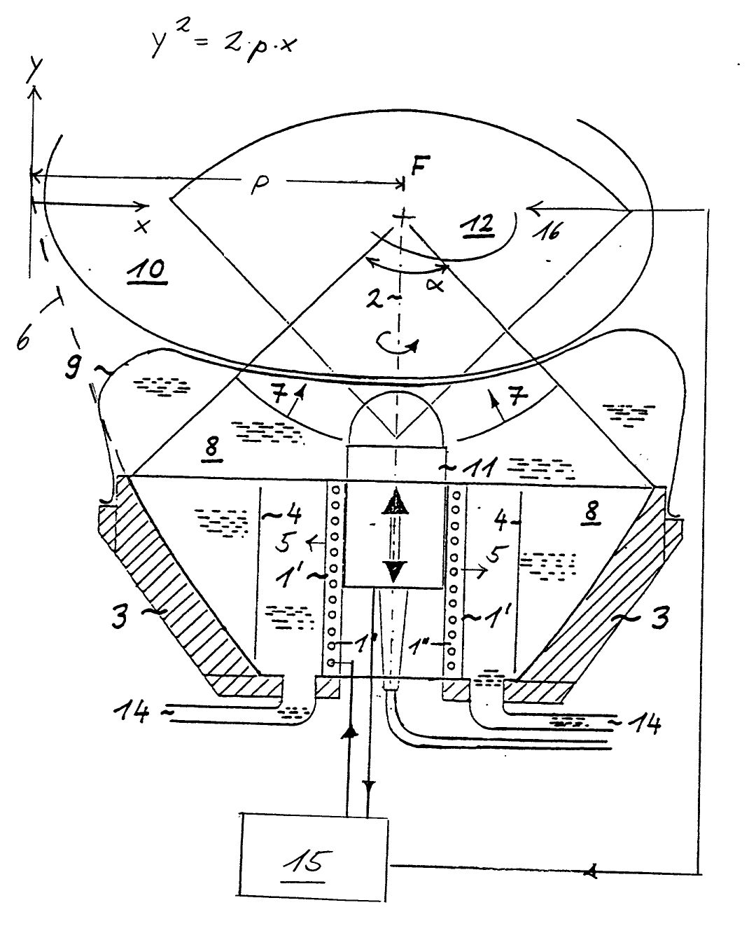

The inventive device excels itself by the provision that at least one shock wave source is used as ultrasonic source, particularly for revascularization and alleviation of pain, which is disposed in extracorporeal arrangement.

### DESCRIPTION

1. Field of the Invention

The present invention relates to a therapeutic method, e.g. for treatment of the heart or pancreas, as well as to a device for performing this method by means of an ultrasonic source generating focussed ultrasonic waves.

2. Prior Art

A therapeutic device for treatment of cardiac diseases and conditions on pericardial vessels is known from the German Patent DE 43 12 2264 A1, wherein therapeutic ultrasonic waves create changes in the tissue by thermal effects. In that device particularly an ultrasonic source is used which is adapted for introduction through the oesophagus.

It is moreover known to use a laser for treatment of the heart, which emits laser pulses which are synchronised in particular with the cardiac rhythm.

### BRIEF DESCRIPTION OF THE INVENTION

In accordance with the present invention it has been found that therapeutic ultrasonic waves, which produce (merely) a thermal effect are suitable for revascularisation and alleviation of pain only to a very restricted extent. Moreover, the introduction of a catheter with the ultrasonic source through the oesophagus is inconvenient for the patient.

On the other hand, the expenditure involved for a “cardiac” laser is very high. Apart therefrom, the treatment with a so-called cardiac catheter, which is necessary for laser application, involves certain risks for the patient.

The invention is now based on the problem of providing a therapeutic device, e.g. for cardiac or pancreatic treatment, which permits an efficient revascularisation or alleviation of pain with a comparatively small expenditure in terms of apparatus and without the introduction of a catheter.

One inventive solution to this problem is defined in Patent claim 1. A device for performing the inventive method is the subject matter of claims 2 et seq.

In accordance with the invention a therapeutic method is proposed which is suitable, for instance, for cardiac treatment or treatment of the pancreas, for instance. The inventive method is particularly well suited for cardiac revascularisation.

The method proposed in the invention uses an ultrasonic source which generates focussed ultrasonic waves. In the inventive therapeutic method the ultrasonic source is at least one shock wave source for extracorporeal arrangement.

As a matter of fact, it has been found in accordance with the invention that an ultrasonic source generating shock or pressure waves, respectively, e.g. of the type used already in lithotripsy, serves to achieve a substantially more efficient revascularisation or alleviation of pain that a an ultrasonic source generating merely thermal effects.

In accordance with the present invention it has been established in particular that—in opposition to the opinion expressed in the technical literature—it is yet well possible to provide for an efficient extracorporeal treatment of a number of organs apparently “covered” or camouflaged by elements of the skeleton such as ribs.

For cardiac treatment, for instance, there is a sufficient number of “windows” in the costal arch for extracorporeal treatment of the heart with ultrasonic waves.

The ultrasonic source(s) used in accordance with the invention may be configured in a form such as that described in the European Patent EP-B-0 369 177 or the German Patent DE 38 35 318 C1, which relates to the same subject matter, and in literature as quoted in both documents. As far as all other terms are concerned which are not described here in greater detail, these prior art documents and the literature quoted therein are referenced expressis verbis here.

In particular, the source of shock waves may comprise an electromagnetic source including at least one coil, as is described in the aforementioned German Patent DE 38 35 318 C21. The coil or coils are preferably disposed on the peripheral surface of a cylinder or a cone and configured, for instance, as cylindrical coils.

With such a coil configuration (at least) one reflector is provided which reflects the ultrasonic waves emitted approximately orthogonally on the axis of the coil unit. This reflector may present a parabolic contour and surround the shock wave source—in this respect reference is equally made to the German Patent DE 38 35 318 C1.

When an enlarged focal region is to be realised it is also possible to substitute a reflector with a parabolic contour by a reflector presenting an ellipsoid shape in at least one section or which is configured as cone with “straight” sections.

In view of the fact that the “acoustic irradiation” with ultrasonic waves must takes place through “windows” in the costal arch for cardiac treatment it is, however, expedient, to use a reflector of a shape other than rotationally symmetrical:

In particular, the surface of the reflector through which the sound emerges may have an elongate shape. The “longer” axis of the sound exit surface is then disposed to extend in “parallel” with the extension of the ribs.

In addition or as an alternative, the coupling cushion, which is disposed between the ultrasonic source and the body, may have an elongate shape so as to support the introduction of the sound waves through “windows” in the costal arch.

The acoustic irradiation through “bone-free” windows in the costal arch is assisted by the provision that at least two ultrasonic sources are provided which are disposed outside the body, whereof (at least) one of the (at least) two ultrasonic sources is a linear ultrasonic transmitter.

Furthermore, at least one locating detector means is preferably provided which is integrated into an ultrasonic source in particular.

The locating detector may be an ultrasonic locating means or an X-ray detector.

It is furthermore preferable to provide a sensor which detects the cardiac action and emits an output signal which is applied to a controller unit.

In order to avoid artefacts due to motion it is expedient that the controller correlates the build-up of the image of the locating detector with the cardiac action detected by the sensor. Correlation can be implemented in particular by means of the R wave.

In another embodiment of the invention the controller correlates the triggering of shock waves with the cardiac action.

### BRIEF DESCRIPTION OF THE DRAWING

The invention will be described in greater detail in the following by an embodiment with reference to the drawing which shows in the single FIGURE

a cross-sectional view taken through an inventive source wave source and a controller.

The FIGURE shows a cross-sectional view of a preferably employed shock wave source as well as the controller unit employed to control the shock wave source.

The shock wave source comprises a sound generator unit 1 for the therapeutic wave field, which has a radiating surface 1′ which, in the illustrated embodiment, has the shape of a cylinder. A reflector 3 is disposed in an arrangement coaxial with the axis 2 of the shock wave source which coincides with the axis of the cylindrical radiating surface 1′.

The reflector 3 may be rotationally symmetrical having an inner surface which presents a parabolic shape in any section. It is moreover possible to use reflectors which have different shapes in both principal sections:

It is possible, for instance, that even though both principal sections have the shape of parabolas that the parabolas are different. It is furthermore possible that one principle section has the shape of a parabola but that the other one has the shape of an ellipse so that an elongate focal area is achieved which is particularly expedient in the cardiac therapy, for instance, on account of the irradiation conditions through the space left open by the ribs.

With the sound generator unit 1 generating cylindrical wave fronts 4—as will be still explained in the following—which move away from the sound generator unit 1 in the radial direction 5, focussed wave fields 7 are created upon reflection at the reflector 3, which has a paraboloid inside contour in particular, with the aperture angle α, which fields converge in the focal point F (more precisely the “focal area”) to generate there power densities which produce therapeutic effects.

The parabola 6, which is present in the illustrated section of the reflector 3 of the embodiment shown here, is defined by the following equation:

y²=2px

wherein p denotes the distance of the focal point F from the co-ordinate origin; the position of the employed co-ordinate system is apparent from the FIGURE.

Explicit reference should be made to the fact that with very high amplitudes of the therapeutic acoustic waves their propagation behaviour may vary from the linear propagation characteristics so that variations may result in focus geometry; these variations in focus geometry may be compensated by an appropriate adaptation of the reflector surface, e.g. by slight variations from the parabolic shape.

It is equally possible to achieve selective variations of the focussed therapeutic wave field by defined variations from the parabolic shape up to the elliptical shape and/or by an appropriate change of the behaviour of the surface in reflection.

Depending on whether a reflection of the wave with or without phase reversal is intended a material is selected for the reflector which is acoustically harder or acoustically softer than the coupling liquid. In the event of generation of predominantly positive pressure pulses it is expedient to use a source which generates positive pressure pulses and an acoustically hard reflector. As a function of the therapeutic objective, however, other source/reflector combinations may be advisable.

In the selection of the reflector material moreover the fact should be considered that—apart from the properties mentioned above already—the propagation velocity for transverse superficial waves will be smaller or only slightly higher than the rate of propagation of the therapeutic waves in the coupling liquid.

In the illustrated embodiment moreover an ultrasonic location detector means 11 is disposed within the cylindrical radiating surface 1′ of the sound generator unit, e.g. in the form of an ultrasonic B-image equipment which serves to represent the focal zone F in the body of an organism 10.

Without any restriction of the general inventive idea, the sound generator unit has moreover been illustrated in an exemplary form as electromagnetic system which comprises a coil 1″ within the radiating surface 1, which may be a cylindrical coil in particular.

For coupling sound waves into the body of organisms it is expedient to generate the waves already in a medium having acoustic characteristics resembling those of tissue, so as to keep the losses in reflection low. Such media may be water, oils or other liquids, for example, which have an acoustic impedance δ*c (density*acoustic velocity) which approaches the impedance of living tissue in the region where the therapeutic wave is coupled in.

Moreover, the reflector 3 is encapsulated by an acoustically transmissive and matched membrane 9. Within the space 8 enclosed by the reflector 3 and the membrane 9 a coupling liquid is contained so as to form a flexible cushion which is acoustically coupled to the body 10 whilst gas pockets are avoided.

The therapeutic waves 4 propagate initially in a radial direction relative to the axis of rotation 2 of the system—as has been described already, and are subsequently reflected by the reflector 3 to the focal point F. The acoustic reflector presents the aforedescribed shape and consists of a material having a high degree of reflectivity relative to the coupling liquid 8.

The acoustic focussed wave field 7 is coupled into the body 10 via the membrane 9 and is directed by means of the ultrasonic locating detector 11 onto the therapeutic target area 12, e.g. the human heart to be revascularised.

The ultrasonic probe 11 is disposed for displacement along the axis 2 in the direction of the arrow for optimisation of the image quality of the ultrasonic image, so that it can be optionally approached to the body or removed therefrom so as to avoid shading or vignetting of the therapeutic wave field. An appropriate position detector ensures a continuous representation of the focal point of the therapeutic field in the ultrasonic image. Furthermore, the ultrasonic probe is arranged for rotation about the axis 2 so that the ultrasonic image can optionally represent different sectional planes.

The therapeutic unit is equipped with connectors 14—in addition to the necessary supply connectors for the therapeutic source and the ultrasonic probe—which are provided for equalising the volume of the coupling cushion by supply or withdrawal of suitably prepared coupling liquids so that an acoustically expedient application of the membrane 9 to the body will be possible. The corresponding supply units such as pumps, controllers, degassing means etc. are not illustrated.

Furthermore a controller unit 15 is provided to which, apart from other data, the output signal from the locating detector means 11 is applied. The output signal of a sensor is also applied to the controller unit 15, which detects the cardiac activity.

The controller unit 15 is capable of correlating, for instance, the build-up of the image of the locating detector means 11 with the cardiac activity detected by the sensor 16 and with the R wave in particular. Moreover, the controller unit is capable of correlating the triggering of shock waves with the cardiac activity.

The present invention has so far been described with reference to a particular embodiment without any restriction of the general inventive idea and without limitation of the general applicability of the invention.

1. Therapeutic method for treatment of the hear or the pancreas, for example, operating on focussed ultrasonic waves,
characterised in that at least one shock wave source is used which is disposed in an extracorporeal arrangement, particularly for revascularisation and alleviation of pain.

2. Therapeutic device for performing the method according to claim 1, comprising an ultrasonic source generating focussed ultrasonic waves,
characterised in that said ultrasonic source is at least one shock wave source disposed in an extracorporeal arrangement, in particular for revascularisation and alleviation of pain.

3. Therapeutic device according to claim 2,
characterised in that said shock wave source is an electromagnetic source including at least one coil.

4. Therapeutic device according to claim 3,
characterised in that said coil or coils are arranged on the peripheral surface of a cylinder, and that a reflector is provided for reflecting the radiated ultrasonic waves.

5. Therapeutic device according to claim 4,
characterised in that said reflector has a parabolic contour.

6. Therapeutic device according to claim 4 or 5,
characterised in that said reflector surrounds said shock wave source.

7. Therapeutic device according to any of the claims 3 to 5,
characterised in that said reflector is not rotationally symmetrical.

8. Therapeutic device according to claim 7,
characterised in that the surface of said reflector through which the sound emerges presents an elongate shape.

9. Therapeutic device according to any of the claims 1 to 8,
characterised in that said coupling cushion, which is disposed between said ultrasonic source and the body, has an elongate shape.

10. Therapeutic device according to any of the claims 1 to 9,
characterised in that at least two ultrasonic sources are provided which are disposed in extracorporeal arrangement.

11. Therapeutic device according to claim 1 0
characterised in that one of said two ultrasonic waves is a linear ultrasonic transmitter.

12. Therapeutic device according to any of the claims 1 to 11,
characterised in that at least one locating detector means is provided.

13. Therapeutic device according to claim 12,
characterised in that said locating detector means is integrated into an ultrasonic source.

14. Therapeutic device according to claim 12 or 13,
characterised in that said locating detector means is an ultrasonic locating detector.

15. Therapeutic device according to claim 12 or 13,
characterised in that said locating detector means is an X-ray locating detector.

16. Therapeutic device according to any the claims 11 to 15,
characterised in that a sensor is provided for detecting the cardiac activity and producing an output signal applied to a controller unit.

17. Therapeutic device according to claim 16,
characterised in that said controller unit correlates the build-up of the image of said locating detector means with the cardiac activity detected by said sensor.

18. Therapeutic device according to claim 17,
characterised in that said controller unit correlates the image build-up with the R wave.

19. Therapeutic device according to any of the claims 15 to 18,
characterised in that said controller unit correlates the triggering of shock waves with the cardiac activity.
